(19) United States
(12) Patent Application Publication
(10) Pub. No.: US 2014/0194648 A1
(43) Pub. Date: Jul. 10, 2014

(54) PROCESS FOR PRODUCING 2-ETHYLHEPTANOIC ACID

(71) Applicants: Christian Boeing, Koeln (DE); Dietrich Maschmeyer, Recklinghausen (DE); Markus Winterberg, Datteln (DE); Michael Grass, Haltern am See (DE); Andreas Gevers, Bottrop (DE); Hinnerk Gordon Becker, Essen (DE)

(72) Inventors: Christian Boeing, Koeln (DE); Dietrich Maschmeyer, Recklinghausen (DE); Markus Winterberg, Datteln (DE); Michael Grass, Haltern am See (DE); Andreas Gevers, Bottrop (DE); Hinnerk Gordon Becker, Essen (DE)

(73) Assignee: Evonik Degussa GmbH, Essen (DE)

(21) Appl. No.: 14/122,041

(22) PCT Filed: May 15, 2012
(86) PCT No.: PCT/EP2012/059011

§ 371 Date: Nov. 25, 2013

(30) Foreign Application Priority Data
Jun. 21, 2011 (DE) 10 2011 077 860.8

(51) Int. Cl. C07C 51/09; C07C 67/08

(52) U.S. Cl. CPC C07C 51/09; C07C 67/08
USPC 560/231; 562/606

(57) ABSTRACT

Process for producing 2-ethylheptanoic acid.

The present invention relates to a process for producing 2-ethylheptanoic acid.

2-Ethylheptanoic acid is known per se. It has properties which make it predestined as a replacement for the 2-ethylhexanoic acid used hitherto on a large scale.

Moreover, the plasticizers produced with 2-ethylheptanoic acid have lower volatility on account of the higher carbon number.

Hitherto, however, the replacement of 2-ethylhexanoic acid by 2-ethylheptanoic acid has not been successful since no production process is known which is able to produce 2-ethylheptanoic acid in high yields, cost-effectively and in a manner suitable for the industrial scale.

Prior art is a production process which is described in WO 02/16301. Here, the lactone 2-ethylidene-6-hepten-5-olide is cleaved to give 2-ethylidenehexenoic acid and then hydrogenated to give 2-ethylheptanoic acid. The starting material 2-ethylidene-6-hepten-5-olide is produced cost-effectively in a known process from two molecules of 1,3-butadiene and one molecule of carbon dioxide (the process is described in Behr and Becker, Dalton Trans. 2006, 4607-4613).

The key step of the process described therein is the cleavage of the 2-ethylidene-6-hepten-5-olide, the hydrogenating cleavage of the lactone being catalyzed with the help of “single site” complexes of metals of sub-group 8 which have been modified with phosphine ligands. The process is carried out by means of a homogeneously dissolved catalyst. Alternatively, the immobilization of the “single site” catalyst on a solid support and also the use of a liquid-liquid two-phase system in which one phase consists of water are proposed as reaction control. In the case of the immobilization of the catalyst on a solid support, the phosphine ligands are modified such that they can be bonded to the support. When using a liquid-liquid two-phase system in which one phase consists of water, the phosphine ligands are modified with groups which effect solubility in water.

Disadvantages of the process described in WO 02/16301 are
1. Complex production of the catalysts
2. High sensitivity of the catalysts to oxygen and water
3. Difficult catalyst separation in the case of a homogeneous reaction procedure
4. Complex modification of the phosphine ligands in the case of immobilization and/or continuous catalyst removal.

An object of the present invention consists in developing a process for producing 2-ethylheptanoic acid which does not have one or more of these aforementioned disadvantages and therefore constitutes an improvement compared to this prior art.

DE 2 003 522 describes a hydrogenolytic cleavage of saturated esters over bifunctional catalysts based on transition metals on a support with acid functions, which, according to the invention, are either ion exchanger resins or crystalline, zeolitic solid-body acids. According to this document, it is particularly advantageous to use a solid-body acid which has an “alkylation index” above 100. This index is defined as the amount (in mmol of alkylate) of propyltoluene which is formed per gram of the acid under standard conditions (100° C., propylene saturated) in one hour.

This process cannot be used for the large-scale cleavage of 3,6-diethyltetrahydropyran-2-one because of the completely unsatisfactory useful life of the catalyst.

A. Behr, V. A. Brehme/Journal of Molecular Catalysis A: Chemical 187 (2002) 69-80 describes, on page 71, the problem of opening the ring of 3,6-diethyltetrahydro-2H-pyran-2-one, which, apart from the keto function, has no other double bonds. The problem could not be solved in the aforementioned article. Consequently, there was the preconception towards 3,6-diethyltetrahydro-2H-pyran-2-one that for precisely this compound no ring-opening reaction can be carried out.

A further object of the invention was thus to overcome the preconception that a ring-opening for that specific compound, 3,6-diethyltetrahydropyran-2-one, is not possible, and to develop exactly such a ring-opening reaction for precisely this compound which could also be transferred to an industrial scale.

The objects were achieved by a process for producing 2-ethylheptanoic acid, involving the following process step:

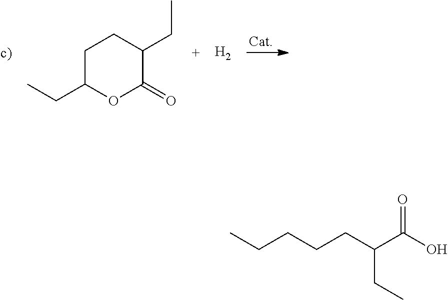

where process step c) is carried out with heterogeneous catalysis over a bifunctional catalyst or over a mixture of at least two catalyst components, where the bifunctional catalyst or, in the case of the mixture, one catalyst component has an acidically acting component, and the acidically acting component comprises a solid oxidic acid which has an alkylation index below 100.

Surprisingly, it has now been found that the cleavage of 3,6-diethyltetrahydropyran-2-one proceeds considerably better with a very high useful life of the catalyst if, instead of a crystalline solid, the solid-body acid used is an amorphous silica-alumina which has an alkylation index below 100.

In a further embodiment of the invention, the 3,6-diethyltetrahydro-2H-pyran-2-one is obtained by the upstream process step b):

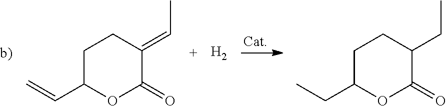

In a further embodiment of the invention, the 2-ethylidene-6-hepten-5-olide is obtained by the upstream process step a):

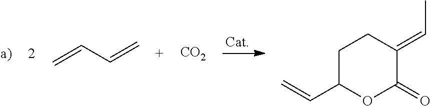

The production of 2-ethylheptanoic acid can thus take place from butadiene and CO₂ by linking together the following process steps:

a. Telomerization of butadiene and CO₂ to give 2-ethylidene-6-hepten-5-olide.

b. Hydrogenation of the 2-ethylidene-6-hepten-5-olide to give 3,6-diethyltetrahydropyran-2-one, preferably over a heterogeneous catalyst on a neutral support, for example palladium on alpha-alumina.

c. Hydrogenolytic cleavage of the 3,6-diethyltetrahydropyran-2-one to give 2-ethylheptanoic acid.

Consequently, it is now possible to produce 2-ethylheptanoic acid on an industrial scale starting from butadiene and CO₂.

In a further embodiment of the invention, the solid oxidic acid in process step c) comprises a substance selected from: amorphous silica-alumina, zirconium dioxide, titanium dioxide. Here, the amorphous silica-alumina is preferred.

Process step c) of the process according to the invention is carried out with heterogeneous catalysis over a catalyst which preferably comprises palladium, platinum or nickel, with palladium being particularly preferred.

The catalyst can be configured in technical terms as a bifunctional catalyst or can consist of a mixture of two catalyst components. Preference is given to using a bifunctional catalyst. It can be produced in such a way that the hydrogenating component is applied to the acidically acting component. This can be carried out by impregnating the acidically acting component with a solution of a salt of the hydrogenating component, or jointly precipitating salts of the acidically acting component and of the hydrogenating component, as described in J. Hagen, Industrial Catalysis: A Practical Approach, Wiley-VCH, Weinheim, 2006. Preference is given to carrying out an impregnation.

Process step c) of the process according to the invention can be carried out in liquid phase, in liquid phase plus gaseous hydrogen phase or in the gas phase. It is preferably carried out in liquid phase with the presence of a gaseous hydrogen phase. In the process step, an organic solvent can be present or the reaction can be carried out in the absence of a solvent. Should a solvent be present, paraffins or ethers, for example, can be used as solvents. Preference is given to carrying out the reaction in the absence of a solvent.

The hydrogenolysis of the lactone 3,6-diethyltetrahydro-2H-pyran-2-one preferably takes place such that 2-ethylheptanoic acid in high yields is formed as product. The alcohols and diols which are often formed during the hydrogenolysis of lactones are undesired in the process according to the invention, and so their formation should be minimized.

Process step c) of the process according to the invention can be carried out at temperatures in the range from 25 to 400° C., preferably in the range from 150 to 350° C.

The pressure can be in the range from 10 to 200 bar, preferably in the range from 20 to 100 bar.

The addition of hydrogen to the reaction mixture can take place in finely divided form and in amounts such that the stoichiometric ratio of hydrogen to the starting material 3,6-diethyltetrahydro-2H-pyran-2-one is between 2 and 1. Preferably, the ratio is between 1.5 and 1.1. It is particularly preferably between 1.2 and 1.1, since otherwise there is the risk that the acid group is hydrogenated to give the alcohol, or the product is further cleaved hydrogenolytically.

The ratio of the mass of the feed stream into the reactor to the mass of the catalyst per hour of residence time [Mfeed/(Vcat*RT), where RT=residence time], known to the person skilled in the art as WHSV (weight hourly space velocity), can, in the case of process step c) of the process according to the invention, be in the range from 0.1 to 20 h⁻¹, preferably in the range from 0.5 to 5 h⁻¹.

Process step c) can be preceded by the following process step b), in which the 3,6-diethyltetrahydro-2H-pyran-2-one is obtained:

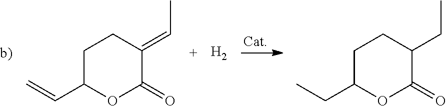

The 3,6-diethyltetrahydro-2H-pyran-2-one obtained here can serve as starting material in subsequent process step c).

Process step b) of a process according to the invention of this embodiment consists of the hydrogenation of 2-ethylidene-6-hepten-5-olide to give 3,6-diethyltetrahydro-2H-pyran-2-one. This process step is preferably carried out with heterogeneous catalysis. For example, the catalyst in process step b) comprises palladium, platinum or nickel. Mixtures of these metals can also be used.

The metals can be used with or without support materials. Should a support material be used, then support materials selected from the group activated carbon, aluminium oxide, silicon oxide, titanium oxide, zirconium oxide or magnesium oxide can be used. Preference is given to using activated carbon or aluminium oxide.

The hydrogenation of 2-ethylidene-6-hepten-5-olide can be carried out in liquid phase, in liquid phase plus gaseous hydrogen phase or in the gas phase. It is preferably carried out in liquid phase with the presence of a gaseous hydrogen phase. In the process step, an organic solvent can be present, or the reaction can be carried out in the absence of a solvent. Should a solvent be present, lower alcohols, paraffins or ethers, for example, can be used as solvent.

The reaction temperature is for example in the range from 0 to 100° C., preferably in the range from 20 to 80° C., particularly preferably in the range from 30 to 70° C.

The pressure is usually in the range from 2 to 50 bar, preferably in the range from 6 to 30 bar, particularly preferably in the range from 10 to 25 bar.

Furthermore, process step c) or process step b) can be preceded by a further process step, process step a). In this, 2-ethylidene-6-hepten-5-olide is obtained by the following reaction:

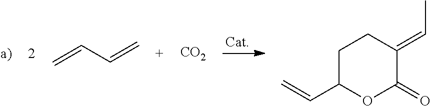

The 2-ethylidene-6-hepten-5-olide obtained in this way can serve as starting material for process step b).

The 2-ethylidene-6-hepten-5-olide is accessible in high yields in a cost-effective manner by virtue of a telomerization reaction of two molecules of 1,3-butadiene and one molecule of carbon dioxide. The process is described inter alia in Behr and Becker, Dalton Trans. 2006, 4607-4613.

In one embodiment of the process, the 2-ethylheptanoic acid obtained in process step c) is reacted in a further process step d) with an alcohol to give an ester. The alcohol can be selected, for example, from:

monohydric, dihydric, trihydric or tetrahydric alcohols, particularly preferably dihydric or trihydric alcohols. Among the di- or trihydric alcohols, particular preference is given, for example, to ethylene glycol, diethylene glycol, triethylene glycol, tetraethylene glycol, propylene glycol, dipropylene glycol, tripropylene glycol, neopentyl glycol, isosorbide, isomannide, isoidide, furan-2,5-dihydroxymethanol, trimethylolpropane, glycerol.

Very particularly preferably, isosorbide and glycerol may be mentioned.

The ester obtained in this way can be used for example as plasticizer, in particular for PVC or PVB.

As well as being used as a starting material for plasticizers, the 2-ethylheptanoic acid produced according to the invention can also advantageously be used in metal salts for use as thermostabilizer and siccative and also for producing lubricants and for producing peroxidic compounds.

The examples below are intended to illustrate the invention in more detail without limiting its implementation to the procedure specified in the examples.

### WORKING EXAMPLES

### Hydrogenation of 2-ethylidene-6-hepten-5-olide to give 3,6-diethyltetrahydro-2H-pyran-2-one (Process step b))

The hydrogenation is carried out in a 3 liter steel autoclave with heating jacket, through which a heat-transfer oil (Marlotherm SH from Sasol Olefins & Surfactants GmbH) flowed. The catalyst used is 5 g of a coated catalyst with 0.5% palladium on alpha-aluminium oxide in bead form which is incorporated into the reactor in a cage such that the mixed gas and liquid phase flows through it in an optimum manner.

152 g (1 mol) of 2-ethylidene-6-hepten-5-olide are introduced into the steel autoclave and dissolved in 11 of tetrahydrofuran. The autoclave is then closed. By injecting hydrogen, a pressure of 20 bar is established. The suspension is held under these conditions at 60° C. for 20 h. The system is then decompressed, the liquid phase is drawn off, the solvent is distilled off on a rotary evaporator and the product is purified by fractional distillation. The yield of 3,6-diethyltetrahydro-2H-pyran-2-one was 145 g (93%).

### Hydrogenolysis of 3,6-diethyltetrahydro-2H-pyran-2-one to give 2-ethylheptanoic acid (Process step c))

The hydrogenolysis is carried out in a tubular reactor (steel 1.4571, internal dimensions 800×8 mm) with a heating jacket, through which a heat-transfer oil (Marlotherm SH from Sasol Olefins & Surfactants GmbH) flowed. The catalyst used is 50 g of pellets (ca. 90 ml) of amorphous silica-alumina with 13% aluminium oxide content and a BET surface area of 290 m²/g which have been impregnated with 2.0% palladium. The reaction temperature is 270° C. The WHSV value based on 3,6-diethyltetrahydro-2H-pyran-2-one is 0.8 kg/I/h based on the empty reactor. In parallel to 3,6-diethyltetrahydro-2H-pyran-2-one, 15 I/h (STP) of hydrogen are fed into the feed of the reactor. The reactor is held at a pressure of 30 bar using argon. The discharge from the reactor is analyzed by gas chromatography. The yield is 89% of 2-ethylheptanoic acid.

1. A process for producing 2-ethylheptanoic acid, the process comprising:
(i) reacting 3,6-diethyltetrahydro-2H-pyran-2-one of formula (I) with hydrogen to obtain 2-ethylheptanoic acid of formula (II) in the presence of a heterogeneous catalyst, 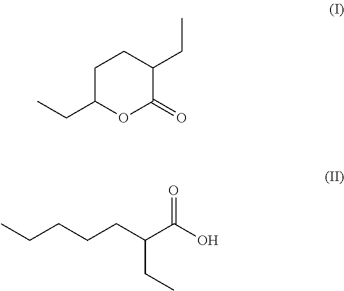
wherein the heterogeneous catalyst is a bifunctional catalyst or a catalyst mixture of at least two catalyst components,
the bifunctional catalyst or one catalyst component of the catalyst mixture has an acidically acting component, and
the acidically acting component comprises a solid oxidic acid which has an alkylation index below 100.

2. The process according to claim 1, further comprising: before said reacting (i),
(ii) reacting 2-ethylidene-6-hepten-5-olide of formula (III) with hydrogen in the presence of a catalyst to obtain 3,6-diethyltetrahydro-2H-pyran-2-one:

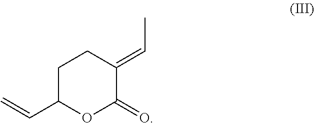

3. The process according to claim 2, further comprising: before said reacting (ii),
(iii) reacting a compound of formula (IV) with carbon dioxide in the presence of a catalyst to obtain 2-ethylidene-6-hepten-5-olide:

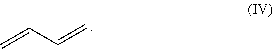

4. The process according to one claim 1,
wherein the solid oxidic acid in said reacting (i) comprises a substance selected from the group consisting of an amorphous silica-alumina, zirconium dioxide, and titanium dioxide.

5. The process according to claim 1,
wherein the heterogeneous catalyst in said reacting (i) comprises palladium, platinum or nickel.

6. The process according to claim 1,
wherein said reacting (i) is carried out in the absence of a solvent.

7. The process according to claim 1,
wherein said reacting (i) is carried out at a temperature of from 25° C. to 400° C.

8. The process according to claim 1,
wherein said reacting (i) is carried out at a pressure of from 1 bar to 200 bar.

9. The process according to claim 1,
wherein said reacting (i) is carried out at a mass ratio of a feed stream to the heterogeneous catalyst per hour of residence time of from 0.1 to 20 h⁻¹.

10. The process according to claim 2,
wherein the catalyst in said reacting (ii) comprises palladium, platinum or nickel.

11. The process according to claim 2,
wherein said reacting (ii) is carried out at a temperature of from 0° C. to 100° C.

12. The process according to claim 2,
wherein said reacting (ii) is carried out at a pressure of from 2 bar to 50 bar.

13. A process for producing an ester, the process comprising:
reacting 2-ethylheptanoic acid with an alcohol to obtain an ester,
wherein the 2-ethylheptanoic acid is obtained by the process according to claim 1.

14. (canceled)
